(19) United States
(12) Patent Application Publication
(10) Pub. No.: US 2026/0363561 A1
(43) Pub. Date: Nov. 19, 2026

(54) CONTINUOUS BLOOD PRESSURE MONITOR

(71) Applicant: Digital & Future Technologies Limited, Carnforth (GB)

(72) Inventor: Christopher John Crockford, Carnforth (GB)

(73) Assignee: Digital & Future Technologies Limited, Carnforth, OT (GB)

(21) Appl. No.: 19/103,331

(22) PCT Filed: Aug. 11, 2023
(86) PCT No.: PCT/GB2023/052126

§ 371 Date: Feb. 12, 2025

(30) Foreign Application Priority Data
Aug. 12, 2022 (GB) 2211858.2
Jun. 9, 2023 (GB) 2308609.3

(51) Int. Cl. A61B 5/021; A61B 5/00; A61B 5/0205; A61B 5/11; A61B 5/352; A61B 5/36

(52) U.S. Cl. CPC A61B 5/021; A61B 5/0006; A61B 5/0022; A61B 5/0205; A61B 5/1118; A61B 5/352; A61B 5/36; A61B 5/4857; A61B 5/6892; A61B 5/706; A61B 5/742; A61B 2562/0219

(57) ABSTRACT

A device for estimating blood pressure from an electrocardiogram (ECG) signal which includes a processor and a memory for storing non-transitory data defining a neural network trained to output a blood pressure estimate from input ECG samples, the processor being configured to implement the neural network on input ECG samples to thereby form an estimate of blood pressure. The device is configured to estimate a QT interval represented in the input ECG samples, and to form the blood pressure estimate in dependence on (a) the estimated QT interval and (b) the ECG samples.

This invention relates to continuous systolic and diastolic blood pressure monitoring. A method and device that uses input ECG data and a neural network to process the data is described.

### BACKGROUND

Blood pressure is a measure of the intra-vascular pressure. Systolic blood pressure is the maximum pressure during a heartbeat, and diastolic blood pressure is the minimum pressure between consecutive beats. High blood pressure (hypertension) exerts strain on the circulatory system, which may cause arterial walls to thicken and become less flexible, or to become weaker and narrowed over time. Weaker and less elastic arteries are susceptible to rupture, leading to stroke or myocardial infarction. Low blood pressure (hypotension) may result in insufficient oxygen delivery and serious shock. Hypotension causes dizziness and light-headedness.

Existing systems for monitoring cardiovascular health use electrocardiogram (ECG) or photoplethysmogram (PPG) techniques to measure pulse signals. In an ECG, electric sensors attached to the skin are used to detect the electrical signals produced by the heart each time it beats. A PPG reading can be obtained through optronics, normally a light-emitting diode (LED) and a photodiode pair operating in green, red or infrared wavelength ranges. The LED illuminates the skin and then the amount of light either transmitted or reflected is measured by the photodiode. During each cardiac cycle, a pressure pulse reaches the skin and distends the arteries and arterioles therein, resulting in a measurable change in light transmission/reflection in the skin.

The most common way to measure blood pressure is with a sphygmomanometer, consisting of an inflatable cuff, measuring unit, and mechanism for inflation. This direct measurement technique has the disadvantage that it is not able to provide continuous measurements and is typically not available outside of clinical settings. There is a need for an improved continuous blood pressure measurement device.

### SUMMARY OF THE INVENTION

According to an aspect there is provided a device for estimating blood pressure from an electrocardiogram (ECG) signal, the device comprising a processor and a memory for storing non-transitory data defining a neural network trained to output a blood pressure estimate from input ECG data, the processor being configured to implement the neural network on input ECG data to thereby form an estimate of blood pressure.

The processor may be configured to filter received ECG data to resolve it into discrete cardiac cycles therein and implement the neural network on the filtered data to thereby form an estimate of blood pressure.

The processor may be further configured to average the ECG data over multiple resolved discrete cardiac cycles and implement the neural network on averaged data.

The number of multiple discrete cardiac cycles may be greater than or equal to ten and less than 100. The discrete cardiac cycles may be aligned with R-R peak intervals.

The processor may be further configured to calculate a QTc timing and normalise the input ECG data by the QTc timing.

The device may be adapted to be worn by a human.

The device may comprise communication hardware for connecting to a smartphone, the communication hardware being arranged to cause an estimated blood pressure value to be output. The communication hardware may be capable of connecting to the smartphone by a wired or wireless data connection. The communication hardware may be configured to receive one or more measurements, process those measurements to estimate a blood pressure value, and transmit that blood pressure value over the data connection.

The device may further comprise communication hardware for connecting to a cloud or to a server at a remote location, wherein the communication hardware is configured to transmit processed data for storage on a cloud. The communication hardware may be capable of supporting a wired or wireless data connection to the internet for transmitting data to the cloud or remote server.

A device according to any preceding claim, wherein the processor is a neural network processing unit. The processor may comprise hardware specifically adapted from neural network and/or machine learning processing.

The device may further comprise a display for showing an estimate of blood pressure.

In a further embodiment there is a device for estimating blood pressure from an ECG signal, the device comprising a processor, a memory for storing non-transitory data defining a neural network trained to output a blood pressure estimate from input ECG data, and a display, the processor being configured to implement the neural network on input ECG data to thereby form an estimate of blood pressure and to cause the estimate to be displayed on the display.

The device may further comprise sensing apparatus for measuring ECG data.

The sensing apparatus for measuring ECG data may be in the form of a mat comprising ECG sensors, the mat having indicia for guiding a user to place a hand in a desired location on the mat relative to the sensors.

The sensing apparatus for measuring ECG data may be in the form of a portable unit.

The portable unit may comprise at least one sensor for contacting a thenar eminence of a user and indicia for guiding a user to grip the portable device. The indicia may be configured to guide the user to grip the portable device with their thenar eminence contacting the sensor.

At least one sensor may be located on the mat so as to be positioned adjacent a thenar eminence of a user when the user's hand is aligned with the indicia.

In a further embodiment the device is communicatively coupled to three or more additional sensors for sensing ECG data.

There is provided a method for estimating blood pressure comprising the steps of receiving ECG data; filtering the received ECG data to resolve it into R-R peak intervals; processing the filtered data using a neural network or by means of a machine learning algorithm; outputting a value of blood pressure.

The method for determining blood pressure may further comprise applying discrete ECG analysis to smooth the outputs of the neural network.

The method for determining blood pressure wherein the discrete ECG analysis involves resolving the data into R peaks and averaging over an integer number of R-R peak intervals and inputting the averaged data before processing.

A method for determining blood pressure is provided wherein the ECG data is normalised by a QTc timing.

The method for determining blood pressure may further comprise the step of assessing the quality of input data before the filtering step.

The method for determining blood pressure may further comprise the step of selecting data from at least one of a plurality of data sources in dependence on the quality of the recorded data.

The method may further comprise the step of measuring ECG data with a mat comprising at least one sensor.

According to an aspect there is provided a device for estimating blood pressure from an electrocardiogram (ECG) signal, the device comprising a processor and a memory for storing non-transitory data defining a neural network trained to output a blood pressure estimate from input ECG samples, the processor being configured to implement the neural network on input ECG samples to thereby form an estimate of blood pressure; wherein the device is configured to estimate a QT interval represented in the input ECG samples, and to form the blood pressure estimate in dependence on (a) the estimated QT interval and (b) the ECG samples.

The estimated QT interval may be a corrected (QTc) interval. The estimated QT interval may be formed by determining a raw QT interval from ECG data and then correcting that raw QT interval for heart rate.

The device may be configured to estimate the QT interval from the ECG samples.

The ECG samples may be samples of data sensed by an electrocardiogram at respective times. Each ECG sample may represent cardiac activity of a subject at the respective time.

The device may store data defining a plurality of neural network algorithms. The device may be configured to select in dependence on the estimated QT interval one of the stored algorithms and use the selected algorithm to form the blood pressure estimate in dependence on the ECG samples.

The device may store a set of ranges of QT values and may be configured to select one of the stored algorithms in dependence on in which of those ranges the estimated QT interval falls.

The device may store data defining a neural network algorithm adapted to take as inputs (a) the estimated QT interval and (b) the ECG samples and to form an estimate of blood pressure therefrom.

The processor may be configured to filter received ECG data to resolve it into discrete cardiac cycles therein. The processor may be configured to implement the neural network on the filtered data to thereby form an estimate of blood pressure.

The device may further comprise an accelerometer configured to measure activity and provide a measurement of activity to the processor. The device may be configured to store data sensed by the accelerometer.

The device may further comprise communication hardware for communicating with a smartphone. The communication hardware may be configured to cause the blood pressure estimate to be output.

The device may further comprise communication hardware for connecting to a cloud, wherein the communication hardware is configured to transmit processed data for storage on a cloud.

The device may further comprise a display for displaying the blood pressure estimate.

In another embodiment there is provided a device for estimating blood pressure from an electrocardiogram (ECG) signal, the device comprising a processor and a memory for storing non-transitory data defining a neural network trained to output a blood pressure estimate from input ECG samples, the processor being configured to implement the neural network on input ECG samples to thereby form an estimate of blood pressure; wherein the device is configured to receive a measurement of the activity level of a subject during a period over which the ECG samples were collected, and to form the blood pressure estimate in dependence on (a) the measured activity level and (b) the ECG samples.

The device may store data defining a neural network algorithm adapted to take as inputs (a) the measured activity level and (b) the ECG samples and to form an estimate of blood pressure therefrom.

In a further aspect, there is provided a device for estimating blood pressure from an electrocardiogram (ECG) signal, the device comprising a processor and a memory for storing non-transitory data defining a neural network trained to output a blood pressure estimate from input ECG samples, the processor being configured to implement the neural network on input ECG samples to thereby form an estimate of blood pressure; wherein the device is configured to estimate a stage of circadian rhythm of a subject during a period over which the ECG samples were collected, and to form the blood pressure estimate in dependence on (a) the estimated stage of circadian rhythm and (b) the ECG samples.

The device may further comprise sensing apparatus for sensing light intensity, the sensing apparatus being configured to provide a measure of light intensity to the processor.

The present invention will now be described by way of example with reference to the accompanying drawings. In the drawings:

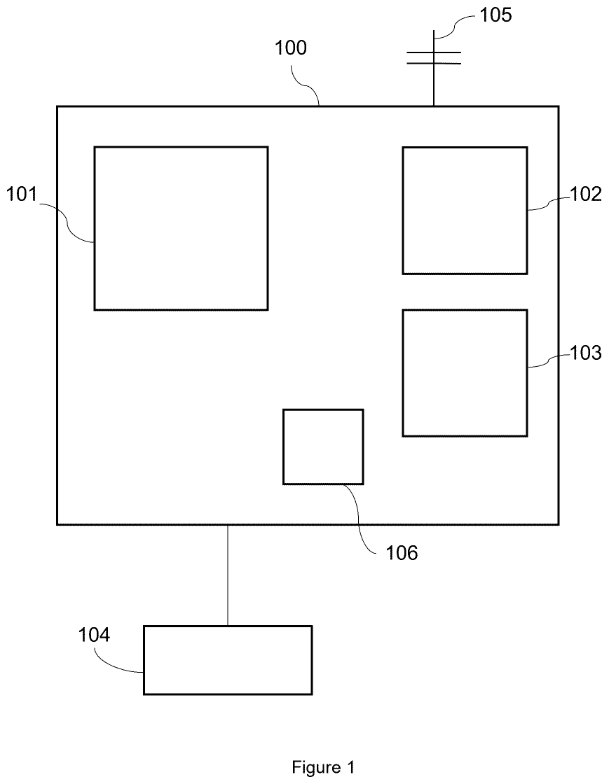

FIG. 1 shows a schematic of the device.

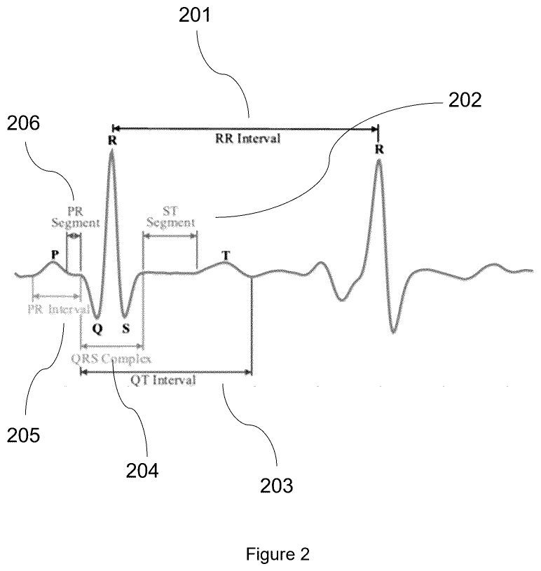

FIG. 2 shows an exemplary ECG signal.

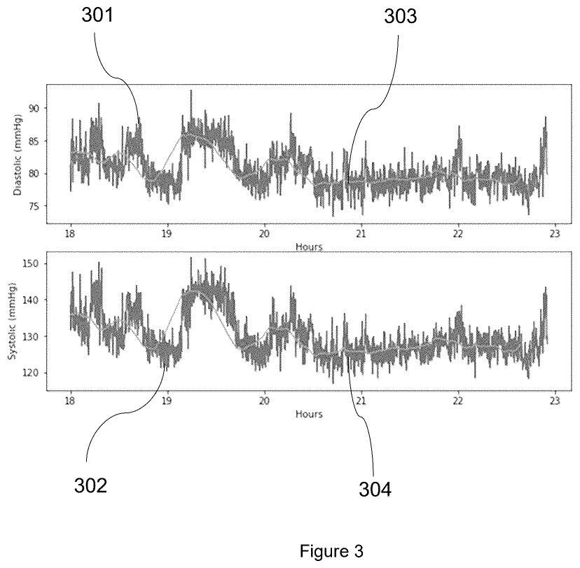

FIG. 3 shows systolic and diastolic blood pressure continuously determined over time.

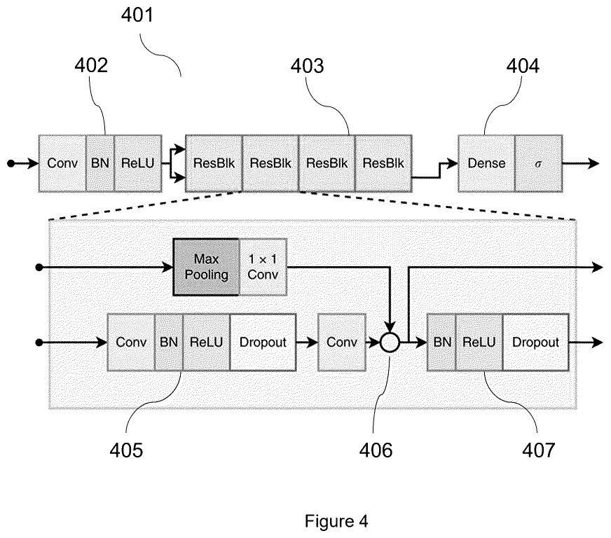

FIG. 4 shows a block diagram of model architecture for a neural network.

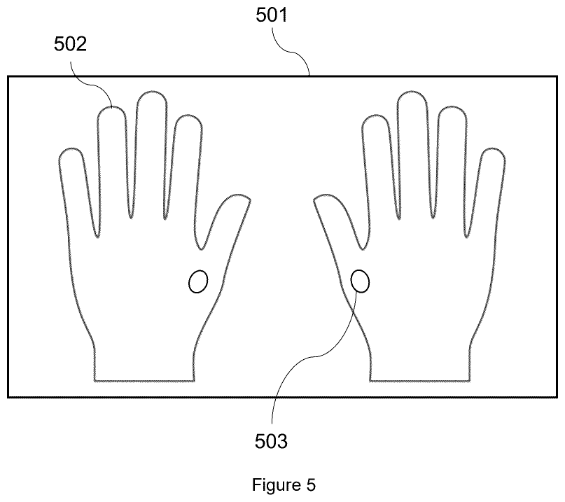

FIG. 5 shows a sensing mat for a user.

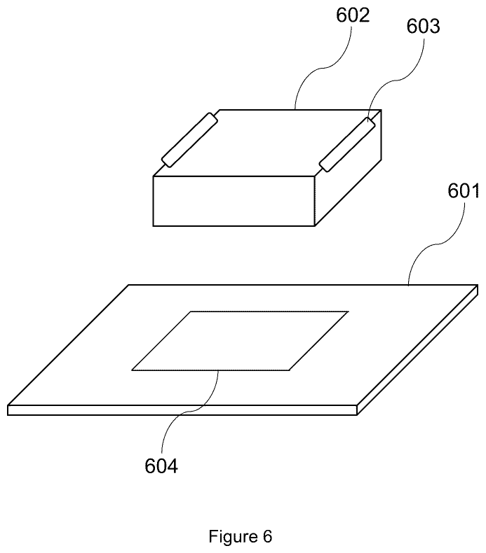

FIG. 6 shows a sensing device for a user.

### DETAILED DESCRIPTION

The following description is presented to enable any person skilled in the art to make and use the invention, and is provided in the context of a particular application. Various modifications to the disclosed embodiments will be readily apparent to those skilled in the art.

The general principles defined herein may be applied to other embodiments and applications without departing from the present invention. Thus, the present invention is not intended to be limited to the embodiments shown, but is to be accorded the widest scope consistent with the principles and features disclosed herein.

FIG. 1 shows an overview of the device 100. The device has a display 101. The device has a processor 102. The device has a memory 103. The memory 103 stores in a non-transitory manner code executable by the processor 102. The device is shown connected to a sensor 104. Alternatively, the sensor 104 may be comprised within the device 100. The device has communication apparatus 105. The device has a movement sensor 106.

The device may receive ECG data. The processor 102 executes code stored on the memory 103. The input ECG data may be filtered by the processor. The ECG data collected may be averaged over an integer number of R peaks. Averaged data may be processed by the processor. The operation of the neural network will be described below with reference to FIG. 4.

The display 101 may be comprised within the device. The display 101 may be separate from the device, for example it may be a smartphone display. The device may have hardware configured to connect to a smartphone and cause an estimated blood pressure value to be displayed. The estimated blood pressure may be two values, one for systolic and the other for diastolic blood pressure. The display may be an OLED, LED, liquid crystal, or other suitable material. The device may be wearable, for example the device may be a wristwatch, have an armband or be a pendant.

The sensor 104 may be an electrode provided at a surface of the device for electrical engagement with the hands of the user. The sensor may be configured to measure an electrocardiogramal. There may be a plurality of sensors 104. The sensor may be positioned in a mat, further described below with reference to FIG. 5. An electrode may be included in the sensor. A stick on electrode that removably adheres to the skin may be included. A sensor system may be a plurality of stick on electrodes.

A movement sensor 106 senses movements and sensed data may be compared with data sensed by the sensor 104. Movement data may be compared with ECG data to filter out low-quality signals. The processor 102 may make a quality assessment of the ECG data using the movement data. The quality assessment may be a pre-processing step before the ECG data is input to the neural network to estimate blood pressure. The movement sensor 106 may be an accelerometer. The accelerometer may be a single-axis or multi-axis accelerometer. The frequency response of the accelerometer may be measured to infer movement of a body. There may be at least one accelerometer.

The communication apparatus 105 may be an antenna. The device may communicate with other local devices in a network using the communication apparatus 105. The communication apparatus may receive sensed data. The communication apparatus may transmit estimated blood pressure values to a storage cloud. The device may transmit data to a user's smartphone.

FIG. 2 shows a theoretical ECG signal. The magnitude of the electric potential of the heart is shown as a function of time. An ECG can detect abnormalities related to heart rhythm and electrical activity and be used to make inferences regarding cardiac structure. The P wave across the PR interval 205 represents depolarisation of the atria, QRS complex 204 represents depolarisation of the ventricles, the T wave represents repolarization of the ventricles, and the ST segment represents when the ventricles are depolarized. The QT interval 203 is the time from when the ventricles start to contract to when they finish relaxing. The QT interval is discerned in ECG samples as the time from the start of the Q wave at the beginning of the QRS complex to the end of the T wave. The start of the Q wave and the end of the T wave may be determined by any suitable method, for example the maximum slope intercept method. The QT interval may be measured over a single heartbeat or averaged (e.g. by taking a mean or median) over multiple beats represented in a set of ECG samples. QT interval is associated with cardiac health and abnormalities may indicate higher risk of cardiovascular disease. The QT interval may be corrected for heart rate by dividing by the square root of the R-R interval to give the QT interval. A prolonged QTc interval is associated with ventricular tachyarrhythmia.

FIG. 3 shows data resulting from a continuous measurement of blood pressures over time. The estimated blood pressure is given as a range, shown by a bar 301 and 302 at each time interval. The calculated value for blood pressure is given by lines 303 and 304 for diastolic and systolic pressure respectively. The description below will describe what will be done with this data. Input data could result from sensor 104. The sensor may be on a wearable device such as a wristwatch. The data resulting from continuous measurements may be shown on a display 101.

FIG. 4 shows a block diagram of a neural network 401. The purpose of the neural network 401 is to estimate blood pressure based on cardiac data in order to provide a continuous and non-invasive measurement of blood pressure more conveniently than by known methods. The neural network implements a machine learning algorithm for estimating blood pressure values, for example systolic and/or diastolic and/or mean and/or median blood pressure.

The neural network receives sensed data and outputs an estimate of blood pressure. The processor may filter or otherwise apply pre-processing transformations to the raw sensed data before it is input to the neural network. The neural network comprises a plurality of layers, each layer being an algorithm that assigns weights to input data.

One approach is to train the neural network with training data. The training data may be collected by a 12-lead ECG assessment with a participant at rest. In a 12-lead ECG, electrodes are placed on the right and left forearms proximal to wrists, right and left shoulders, right and left lower legs proximal to ankles and chest, and six electrodes are arranged on the chest.

Another approach to train the neural network is using a combination of ECG assessment and at least one accelerometer positioned on a participant. The participant is able to move, for example the participant could undertake exercise (e.g. by running on a treadmill) or could carry out normal daily activities, while ECG data is captured. Data from the at least one accelerometer may be correlated to the ECG data, for example by synchronising the time of measurement, to relate activity of a participant to electrocardiogram data. The neural network can be trained on this data in order to learn a relationship between blood pressure and levels of activity. Subsequently, as will be described below, the activity of a participant can be measured and provided as input to the trained neural network together with data providing an estimate of raw blood pressure, and the neural network can output an adjusted estimate of blood pressure that is moderated by the sensed activity level.

In one example, the network inputs ECG data in microvolts to block 402. In block 402 batch normalisation (BN) is applied and a rectified linear activation (ReLU) is used, adopting a pre-activation design. The network has a plurality of layers 403, the layers may be convolutional layers. Shortcut connections such as residual network architecture may be used to optimise tractability. In one example the network architecture has 17 layers and the layers have a filter width of 16. Each layer may has a node 406. Dropout in block 405 is used between the convolutional layers 403 and, in block 407, after the nonlinearity with a pre-set probability. Dropout is applying a filtering step. The final fully connected linear layer produces a value for both systolic and diastolic blood pressure, this is output to block 404.

In an exemplary initiation of the neural network, a random initialisation is used. The Adam optimiser is used with the default parameters β₁=0.9 and β₂=0.999, and a mini-batch size of 32. The exponentially weighted average of the gradients is used to make the algorithm converge at a faster rate. The loss function is a measure of the difference between estimated blood pressure and measured blood pressure. In the following equations:

mₜ=aggregate of gradients at time t (initially, mₜ=0)

mₜ₋₁=aggregate of gradients at time t−1

wₜ=weights at time t

wₜ₊₁=weights at time t+1

αt=learning rate at time t

∂L=derivative of loss function

∂wₜ=derivative of weights at time t

β=moving average parameter

wₜ₊₁=wₜ−αmₜ

where

$${m}_{t} = β {m}_{t - 1} + ( 1 - β ) [ \frac{δ L}{δ {w}_{t}} ]$$

The learning rate is initialised to 10⁻³. The learning rate is reduced by a factor of 10 when the test loss does not improve for two consecutive epochs. A model is chosen that achieves the lowest error on the test dataset. The hyper-parameters of the network architecture and optimisation algorithm may be chosen via manual tuning. The model parameters are adjusted to lower the difference between predictions and blood pressure measurements made directly by sphygmomanometer. The adjustment procedure may be repeated for all ECGs in a training set, using each ECG multiple times.

Once the network has been trained in the manner described above, the parameters may be stored in a memory and provided to a processor. The processor can then be used to estimate blood pressure.

The ECG data may be resolved into packets over a discrete time period. The ECG data may be parameterised. The parameterised data may be used as an input to the neural network to estimate blood pressure for the discrete time period. The R-R peak interval may be used to parameterise the ECG data input. The R-R peak interval corresponds to one cardiac cycle, i.e. a ‘heartbeat’. An integer number of R peaks may be selected and the ECG data averaged over this period before determining a blood pressure. The integer number of R peaks may be between 10 and 100. Alternatively, the ECG data may be normalised by the QTc timing. A combination of R peaks and QTc timing may be used to process the ECG data.

In more detail, in a first embodiment that makes use of QT normalisation, the processing device may have multiple pre-trained neural network algorithms available to it for estimating blood pressure in dependence on a set of measurements. In order to estimate blood pressure from those measurements the device may first select one of those algorithms in dependence on a QT value measured during the period over which the set of measurements was collected, or a QTc value determined form that QT value. Then the device may use the selected algorithm to estimate blood pressure from that set of measurements. IN a second embodiment, the device may have available to it a pre-trained neural network algorithm which takes as one of its inputs a value for QT or QTc, in addition to the set of other measurements. The algorithm may use QT or QTc as an input for estimating blood pressure.

Thus, inputs to the neural network or machine learning algorithm for estimating blood pressure may comprise (a) quantised ECG data measured over a period of time and (b) a QT or QTc value estimated from that ECG data. It may be a mean or median QT or QTc value determined over all that data, or it may be an instantaneous QT or QTc value for a heartbeat during the period represented by the data. Alternatively, the QT or QTc value may be used to select a neural network or machine learning algorithm for estimating blood pressure from the ECG data. The QT value may also be known as a QT interval.

QTc can be estimated from a raw QT measurement using a known function such as the Bazett formula, the Fridericia formula, the Framingham formula or the Hodges formula.

It has been found that by explicitly incorporating QT or QTc as an input to the blood pressure estimation the accuracy of the blood pressure estimation can be improved. Conveniently, multiple models may be pre-trained, each being associated with a range of QT or QTc values, and the device may select one of those algorithms in dependence on a currently measured or estimated QT or QTc value. The measured or estimated QT or QTc value may change over time. It has been found that by estimating blood pressure using a neural network or machine learning algorithm specific to or dependent on a QT or QTc value or a range thereof, blood pressure estimates can be formed that are more reliably comparable over time.

The ECG data may be resolved into packets. Each packet may carry data representing measurements during a respective discrete time period. The measurements may relate to the activity of a person. For example, an accelerometer can be used to determine the body pose and activity level of a person; by supplying acceleration data to a processor, whether the person is supine, sitting, standing or moving can be determined. The packets may be categorised with a certain activity (such as ‘standing’) and the processing of each category of packet may differ. The ECG data recorded during an active period may require more refinement to filter unwanted noise than ECG data recorded while a person is in a supine pose. The accelerometer may be a three-axis accelerometer.

FIG. 5 shows one option for sensor 104. FIG. 5 shows a sensing mat 501 with indicia 502 for guiding a user to place their hands on the mat. A sensor 503 is positioned to align with a thenar eminence of the user's hand. The mat may comprise a plurality of sensors. The sensors may be positioned to align with the fingertips of a user. The sensor may be a movement sensor. The sensor may be an electrode.

The mat may comprise indicia 502 indicating an intended position for the user's hands on the device, a set of electrodes being arranged relative to the set of indicia such that, in use when the user's hands are in the intended position, at least one electrode 503 is located under the thenar eminence of each of the user's hands.

The mat may be connected to a device with a processor configured to process signals in dependence on the output of the one or more movement sensors so as to filter variations from the signals due to movement of the user's hands. The processor may be configured to perform filtering of signals from electrodes in dependence on the output from the respective movement sensor.

FIG. 6 shows an alternative option for sensor 104. FIG. 6 shows a mat 601. The mat 601 may comprise indicia (not shown) for guiding a user to place their hands on the mat. A portable device 602 comprising at least one sensor 603 is shown. The portable device 602 may have indicia (not shown) for guiding a user to grip the device with their hands. The indicia may be hand markings. The indicia may be on opposing sides of the device 602. The indicia may extend around the contours of the device in three dimensions. The indicia may be positioned to align the at least one sensor 603 with a thenar eminence of the user's hand. The at least one sensor may be positioned relative to the indicia to align with the fingertips of a user. The device 602 may be positioned in and retained by the mat 601. The mat 601 may be provided with a recessed area 604. The sensor 603 may be an electrode.

### Circadian Rhythm

A person's blood pressure typically varies during the course of a day. Typically the person's blood pressure is lower overnight, gradually increases after waking, and gradually decreases in the evening. This can cause instantaneous measurements of a person's blood pressure to be unreliable. Measurements of blood pressure taken at different times of the day can give different results. Another consequence of this variation is that medication to reduce or increase blood pressure can be more effective if taken at a certain time of the day. For example, medication to decrease blood pressure might be found to be most effective when taken in the early morning, so that the medication is absorbed into the body and has the highest activity as a person's blood pressure is reaching its diurnal peak.

A body-worn sensor device may estimate a wearer's circadian rhythm from the wearer's behaviour during a day or more preferably over multiple consecutive days. The device may, for example, sense variation in the wearer's heart rate over a day. An extended period of relatively low and/or stable heart rate may indicate that the wearer is sleeping. The period associated with sleeping may, for example, be a period of at least three hours averaged over which the wearer's heart rate is the lowest or the most stable during a 24 hour period. The device may, for example, sense movement. An extended period of relatively little movement may indicate that the wearer is sleeping. The period associated with sleeping may, for example, be a period of at least three hours averaged over which the wearer's motion is the lowest during a 24 hour period. The device may, for example, sense ambient light. An extended period of darkness may indicate that the wearer is sleeping. The period associated with sleeping may, for example, be a period of at least three hours averaged over which the ambient light is the lowest during a 24 hour period.

Human circadian rhythm tends to change relatively slowly. The device may estimate the centre time of a wearer's sleeping period during a day. The device may store an aggregate centre time derived from sensed data on previous days. It may update the aggregate centre time by adjusting it towards the estimated centre time by up to a predetermined amount. The predetermined amount may, for example, be an hour. This can provide a time-smoothed centre time whose daily variation is limited to help model typical human response. That centre time can be used as a marker for the phase of the wearer's circadian rhythm relative to the time of the day. Other methods of estimating the phase of the wearer's circadian rhythm may be used. The wearable device may receive an estimate of the phase of the wearer's circadian rhythm from another source. For example, the another source may be a wristwatch having a light sensor and/or movement sensor.

The wearable device may estimate the wearer's instantaneous blood pressure. It may for example do this by receiving sensed data, processing the sensed data using a trained neural network and outputting an estimate of blood pressure. The device may comprise a processor. The processor of the device may filter or otherwise apply pre-processing transformations to raw sensed data before it is input to the neural network. The neural network comprises a plurality of layers, each layer being an algorithm that assigns weights to input data. The neural network may be trained using training data as described above.

The wearable device may perform this estimation of blood pressure continuously or at intervals of less than an hour or less than 30 minutes or less than 10 minutes. This can provide an indication of the wearer's blood pressure over the course of a day. This information may be used in a number of ways.

In an exemplary use of the information, the blood pressure as measured by the wearable device may be adapted in a predetermined manner in dependence on the state of the user's circadian rhythm at the time the measurement was taken. The adaptation may, for example, be a corrective function applied to the estimated instantaneous blood pressure values. That approach can provide an indication of blood pressure that is adjusted for the expected natural variation in the wearer's blood pressure over a day. The wearable device can perform that adaptation. It can display an estimate of blood pressure that is adapted in that way, to provide an indication to the wearer that is more readily compared with other readings as a result of having been adjusted for circadian rhythm.

In a further exemplary use of the information, the blood pressure as measured by the wearable device over the course of one or more days can be used to adjust the estimate of the phase of the wearer's circadian rhythm. Over time, the device may learn the typical relationship between blood pressure and circadian rhythm for a particular wearer. By comparing that relationship with the unadjusted blood pressure as measured over a day, changes in circadian rhythm can be detected.

In a further exemplary use of the information, the unadjusted blood pressure together with the estimate of the phase of the user's circadian rhythm can be used to estimate when to recommend that the wearer takes medication. One way to do this is to employ a predetermined algorithm to determine a time to recommend, taking the unadjusted blood pressure and the phase of circadian rhythm as inputs. The algorithm may be selected in dependence on the medication for which a time is to be recommended. Another approach is for the device to learn the typical effect of the medication on the wearer's blood pressure. The wearer indicates to the device the time at which they have taken medication. The device can then assess the difference between the measured blood pressure of the wearer after taking the medication and a stored record of the wearer's typical variation in blood pressure during that period. The device may estimate a time (tm) between the taking of the medication and the time, within a window of a predetermined duration such as three, four or six hours, after taking the medication that the maximum difference exists between the wearer's expected blood pressure and their blood pressure after taking the medication. Subsequently the device may recommend taking the medication that time tm before an expected peak low or high value of the wearer's blood pressure.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein. The applicant indicates that aspects of the present invention may consist of any such individual feature or combination of features. In view of the foregoing description, it will be evident to a person skilled in the art that various modifications may be made within the scope of the invention.

1. A device for estimating blood pressure from an electrocardiogram (ECG) signal, the device comprising: a processor and a memory for storing non-transitory data defining a neural network trained to output a blood pressure estimate from input ECG samples, the processor being configured to implement the neural network on input ECG samples to thereby form an estimate of blood pressure; wherein:
the device is configured to estimate a QT interval represented in the input ECG samples, and to form the blood pressure estimate in dependence on (a) the estimated QT interval and (b) the ECG samples.

2. The device as claimed in claim 1, wherein the QT interval is a corrected (QTc) interval.

3. The device as claimed in claim 1 or 2, wherein the device stores data defining a plurality of neural network algorithms, and the device is configured to:
select in dependence on the estimated QT interval one of the stored algorithms; and
use the selected algorithm to form the blood pressure estimate in dependence on the ECG samples.

4. The device as claimed in claim 3, wherein the device stores a set of ranges of QT values and is configured to select one of the stored algorithms in dependence on in which of those ranges the estimated QT interval falls.

5. The device as claimed in claim 1 or 2, wherein the device stores data defining a neural network algorithm adapted to take as inputs (a) the estimated QT interval and (b) the ECG samples and to form an estimate of blood pressure therefrom.

6. The device as claimed in claim 1, wherein the processor is configured to filter received ECG data to resolve it into discrete cardiac cycles therein and implement the neural network on the filtered data to thereby form an estimate of blood pressure.

7. The device according to claim 1, wherein the processor is further configured to average the ECG data over multiple resolved discrete cardiac cycles and implement the neural network on averaged data.

8. The device according to claim 6 or 7, wherein the discrete cardiac cycles are aligned with R-R peak intervals.

9. (canceled)

10. (canceled)

11. The device according to claim 1, further comprising communication hardware for communicating with a smartphone, the communication hardware being configured to cause the blood pressure estimate to be output.

12. The device according to claim 1, further comprising communication hardware for connecting to a cloud, wherein the communication hardware is configured to transmit processed data for storage on a cloud.

13. The device as claimed in claim 1, further comprising a display for displaying the blood pressure estimate.

14. A device for estimating blood pressure from an electrocardiogram (ECG) signal, the device comprising: a processor and a memory for storing non-transitory data defining a neural network trained to output a blood pressure estimate from input ECG samples, the processor being configured to implement the neural network on input ECG samples to thereby form an estimate of blood pressure; wherein:
the device is configured to receive a measurement of the activity level of a subject during a period over which the ECG samples were collected, and to form the blood pressure estimate in dependence on (a) the measured activity level and (b) the ECG samples.

15. The device as claimed in claim 14, wherein the device stores data defining a neural network algorithm adapted to take as inputs (a) the measured activity level and (b) the ECG samples and to form an estimate of blood pressure therefrom.

16. (canceled)

17. (canceled)

18. A device for estimating blood pressure from an electrocardiogram (ECG) signal, the device comprising: a processor and a memory for storing non-transitory data defining a neural network trained to output a blood pressure estimate from input ECG samples, the processor being configured to implement the neural network on input ECG samples to thereby form an estimate of blood pressure; wherein:
the device is configured to estimate a stage of circadian rhythm of a subject during a period over which the ECG samples were collected, and to form the blood pressure estimate in dependence on (a) the estimated stage of circadian rhythm and (b) the ECG samples.

19. The device as claimed in claim 18, further comprising sensing apparatus for sensing light intensity, the sensing apparatus being configured to provide a measure of light intensity to the processor.

20. The device according to claim 1 or claim 14 or claim 18, further comprising an accelerometer configured to measure activity and provide a measurement of activity to the processor.

21. The device according to claim 1 or claim 14 or claim 18, further comprising sensing apparatus for measuring ECG data.

22. The device according to claim 21, wherein the sensing apparatus for measuring ECG data is in the form of a mat comprising ECG sensors, the mat having indicia for guiding a user to place a hand in a desired location on the mat relative to the sensors.

23. The device according to claim 22, wherein at least one sensor is located on the mat so as to be positioned adjacent a thenar eminence of a user when the user's hand is aligned with the indicia.

24. The device according to claim 21, wherein the sensing apparatus for measuring ECG data is in the form of a portable unit, the portable unit comprising at least one sensor for contacting a thenar eminence of a user and indicia for guiding a user to grip the portable device.
